(19) United States
(12) Patent Application Publication
(10) Pub. No.: US 2004/0096396 A1
(43) Pub. Date: May 20, 2004

(54) Compositions and methods for diagnosis and therapy of medical conditions involving angiogenesis

(75) Inventors: John L. Magnani, Gaithersburg, MD (US); John T. Patton JR., Gaithersburg, MD (US)

Correspondence Address: SEED INTELLECTUAL PROPERTY LAW GROUP PLLC, 701 FIFTH AVE, SUITE 6300, SEATTLE, WA, 98104-7092, US

(73) Assignee: GlycoMimetics, Inc., Rockville, MD (US)

(21) Appl. No.: 10/601,080

(22) Filed: Jun. 19, 2003

Related U.S. Application Data
(60) Provisional application No. 60/393,577, filed on Jul. 3, 2002.

(51) Int. Cl. A61K 49/00

(52) U.S. Cl. 424/9.6

(57) ABSTRACT

Compositions and methods are provided for diagnosis or therapy of medical conditions, or for tissue engineering, involving angiogenesis accompanied by the expression of E-selectin. More specifically, compounds are used that are selective for E-selectin binding with no significant ability to bind to P-selectin.

### BACKGROUND OF THE INVENTION

1. Field of the Invention

The present invention relates generally to compositions and methods for diagnosis or therapy of medical conditions, or for tissue engineering, involving angiogenesis accompanied by the expression of E-selectin. More specifically, the invention relates to the use of compounds selective for E-selectin binding with no significant ability to bind to P-selectin. These compounds can be linked to an agent(s) useful for diagnosis or therapeutic intervention of the disease, or to facilitate successful tissue engineering.

2. Description of the Related Art

In human diseases or tissue engineering involving or related to the vasculature system, angiogenesis is required for the progression of the disease or successful application of tissue engineering. Angiogenesis is the biological process of development and growth of new blood vessels to areas undergoing tissue repair and remodeling, or to areas of expanding disease. Angiogenic responses include both normal and pathological processes. Normal states involving angiogenesis include the growth of new blood vessels in response to wound injury (and thus subsequent healing) and in the neovascularization associated with ischemic injury and vascular blockage. An example of a pathological angiogenic process is the development of new blood vessels for tumor growth and subsequent metastasis. In solid tumor cancers, a tumor can grow to the size of about 1 mm diameter without the need of separate blood supply to support its metabolism. Beyond 1 mm diameter, a tumor requires its own vasculature system to support expanded growth. During this phase of cancer progression, neovasculatization occurs at the site of the tumor with the expression of E-selectin in this newly formed vasculature. Indeed, angiogenesis and its related rate of vascular development are a prognostic marker for tumor metastasis and survival.

In tissue engineering, the goal is to design and develop biological replacements for diseased or damaged tissues and organs. These replacements can provide structural functionality (e.g., bone, cartilage, or skin replacements) or metabolic functionality (e.g., liver, pancreas, kidney). This requires the generation of matrices of cells and extracellular components organized in an appropriate way to restore the function of the tissue or organ that is to be replaced. Current approaches include seeding relevant cells on scaffolds or matrices prior to implantation in a patient. For these tissue engineering applications, neovascularization into the scaffold or matrices containing cells is required to achieve the engineered functionality of the new tissue. The ability to promote angiogenesis in the new tissue to create a viable and functioning vasculature system compatible with the patient is a challenge for the field of tissue engineering.

Selectins are a group of structurally related cell surface receptor proteins that are important for mediating adhesion to endothelial cells. These proteins are type 1 membrane proteins and are composed of an amino terminal lectin domain, an epidermal growth factor (EGF)-like domain, a variable number of complement receptor related repeats, a hydrophobic domain spanning region and a cytoplasmic domain.

E-selectin and P-selectin are expressed on the surface of activated endothelial cells, lining the interior walls of the vasculature system. E-selectin binds the carbohydrate sialyl-Lewis^{x} (SLe^{x}), which is presented as a glycoprotein or glycolipid on the surface of certain leukocytes (monocytes and neutrophils), and helps these cells adhere to vasculature walls. E-selectin also binds sialyl-Lewis^{a} (SLe^{a}), which is expressed on many tumor cells. P-selectin is expressed on inflamed endothelium and also on platelets. P-selectin recognizes SLe^{x} and SLe^{a}, but also contains a second site that interacts with sulfated tyrosine containing proteins and peptides. The expression of E-selectin and P-selectin is generally increased when the tissue adjacent to a capillary is infected or damaged.

In the context of angiogenic diagnostics and therapeutics, there is a need to develop highly specific diagnostics and effective clinical intervention that are highly specific to the site of angiogenesis without detrimental side effects to healthy and normally functioning areas of the vasculature system. Due to the difficulties in the current approaches in the art, there is a need for improved compositions and methods.

### BRIEF SUMMARY OF THE INVENTION

Briefly stated, this invention provides compositions and methods for utilizing E-selectin expressed in the neovasculature for the diagnosis and therapy of medical conditions involving angiogenesis and for applications of tissue engineering. The present invention fulfills the need to develop approaches that are effective and highly specific by focusing on the use of E-selection expression (e.g., upregulated) in the neovasculature as a target for binding and inhibition or promotion. In the present invention, compounds highly specific to E-selectin may be used in diagnostics and therapeutics for diseases requiring or associated with angiogenesis. Specific binding of E-selectin is critical in the clinical intervention in these diseases, in order to avoid potential side effects resulting from P-selectin expression, e.g., on platelets.

In one embodiment of the present invention, a method is provided for screening in vivo for a condition requiring or associated with angiogenesis, comprising the steps of: (a) administering to a warm-blooded animal in a diagnostically effective amount any one of compounds 1-15 of FIG. 1; and (b) detecting the compound in the animal. In another embodiment, a method is provided for screening in vitro for a condition requiring or associated with angiogenesis, comprising the steps of: (a) contacting a biological preparation with a diagnostically effective amount of any one of compounds 1-15 of FIG. 1; and (b) detecting the compound in the preparation. In another embodiment, a method is provided for in vitro identification of cells expressing E-selectin, comprising the steps of: (a) contacting a biological preparation with any one of compounds 1-15 of FIG. 1; and (b) detecting the compound in the preparation. In another embodiment, a method is provided for treating a condition requiring or associated with angiogenesis, comprising the step of administering to a warm-blooded animal in a therapeutically effective amount any one of compounds 1-15 of FIG. 1. In another embodiment, a method is provided for promoting angiogenesis in tissue engineering, comprising the step of contacting cells with any one of compounds 1-15 of FIG. 1, wherein the compound possesses an angiogenesis promoting agent.

In an embodiment of the present invention, conjugates are provided. The conjugates comprise any one of compounds 1-15 of FIG. 1 covalently attached to a diagnostic or therapeutic agent. Preferred therapeutic agents include antineoplastic agents, angiogenesis promoting agents and angiogenesis inhibiting agents.

An E-selectin compound described herein or conjugate thereof may be used in a variety of methods. Such uses include: in a method of screening in vitro or in vivo for a condition (e.g., medical condition) requiring or associated with angiogenesis; in a method of treating a condition requiring or associated with angiogenesis; in a method for inhibiting angiogenesis; and in a method for promoting angiogenesis (e.g., tissue engineering). The E-selectin compounds or conjugates thereof may also be used in the manufacture of a medicament, such as for treating a condition requiring or associated with angiogenesis, for inhibiting angiogenesis, or for promoting angiogenesis.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIGS. 1A-D shows the structures of E-selectin specific compounds.

FIG. 2 shows the structures of selectin compounds (which are related to the structures in FIG. 1) that demonstrate P-selectin binding in an ELISA assay.

FIG. 3 depicts the synthesis of representative E-selectin compound 3.

FIG. 4 depicts the synthesis of compound 21.

FIG. 5 depicts the synthesis of representative E-selectin compound 15.

FIG. 6 depicts the acylation of compound 21 to give a variety of representative E-selectin compounds.

FIG. 7 is a table showing the activity of all the compounds of FIGS. 1 and 2 for E-selectin and P-selectin in ELISA assays.

### DETAILED DESCRIPTION OF THE INVENTION

As noted above, the present invention has identified compounds that are highly specific for E-selectin and not for P-selectin for use in diagnostics and therapeutics of medical conditions (including human disease) involving angiogenesis and for applications of tissue engineering.

### E-Selectin Compounds

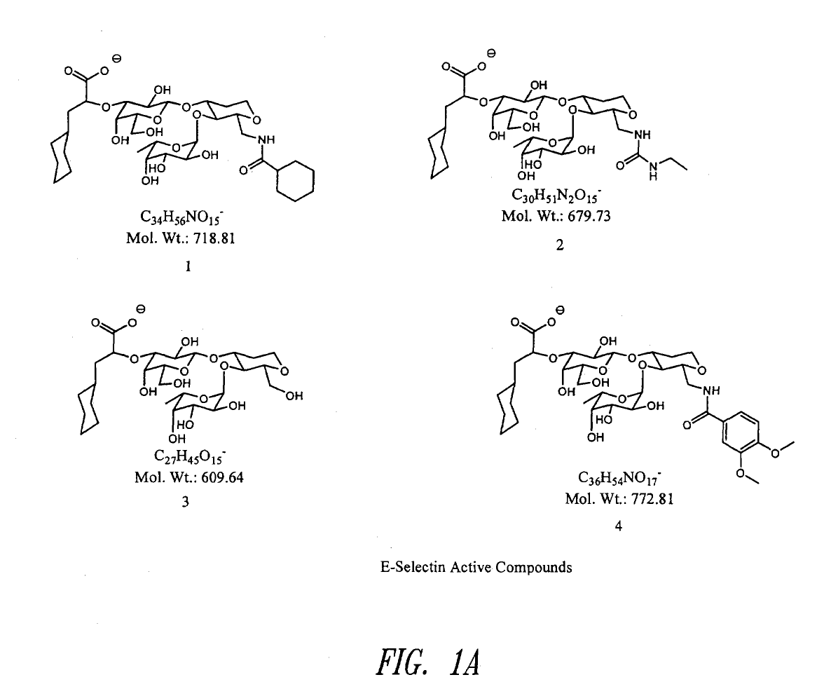

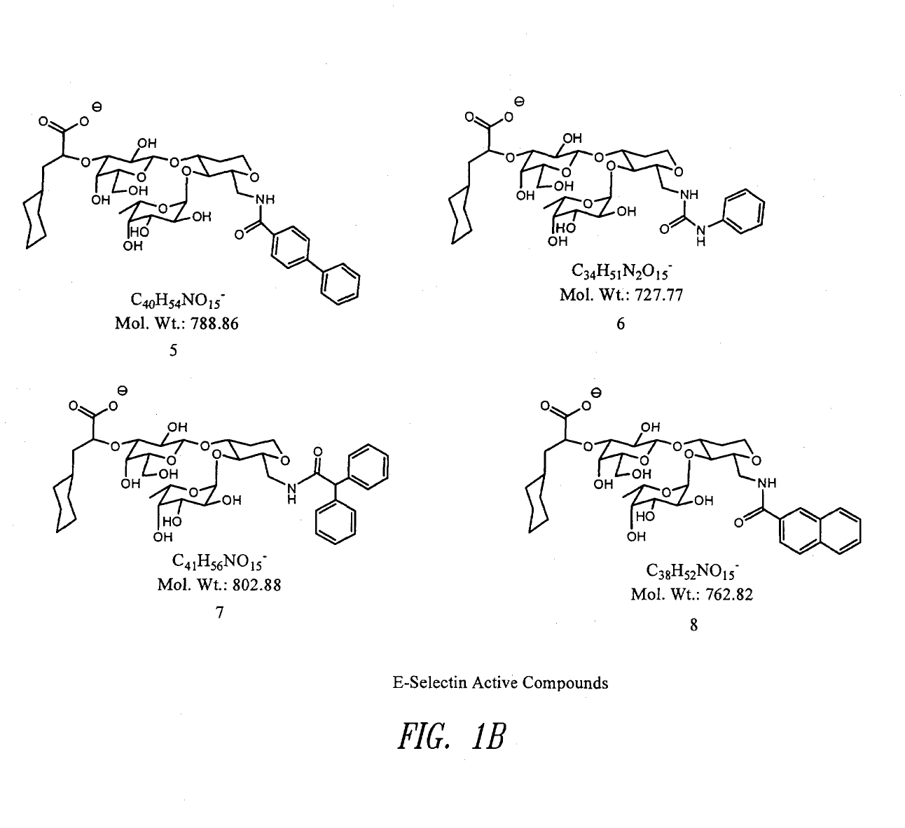

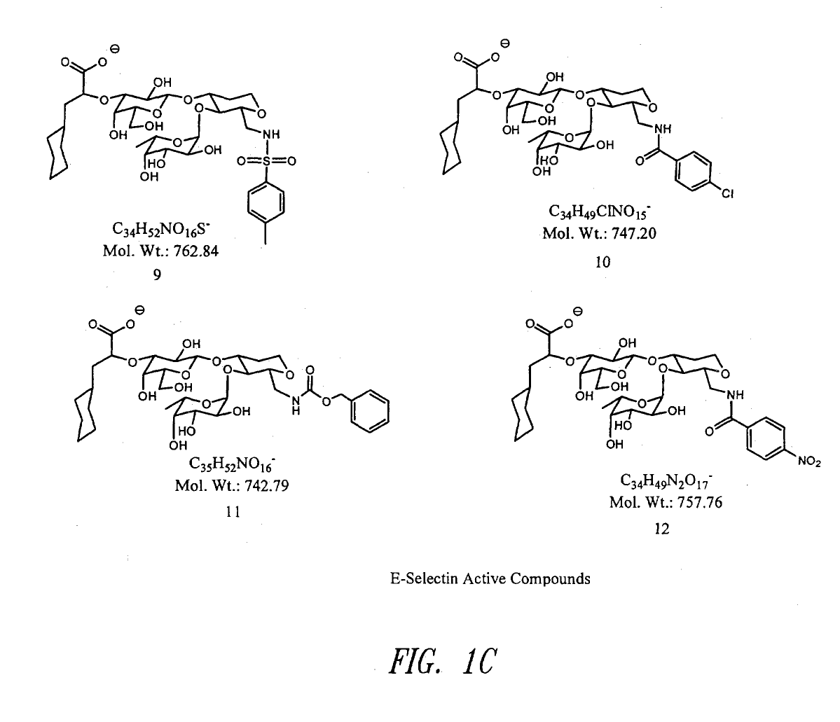

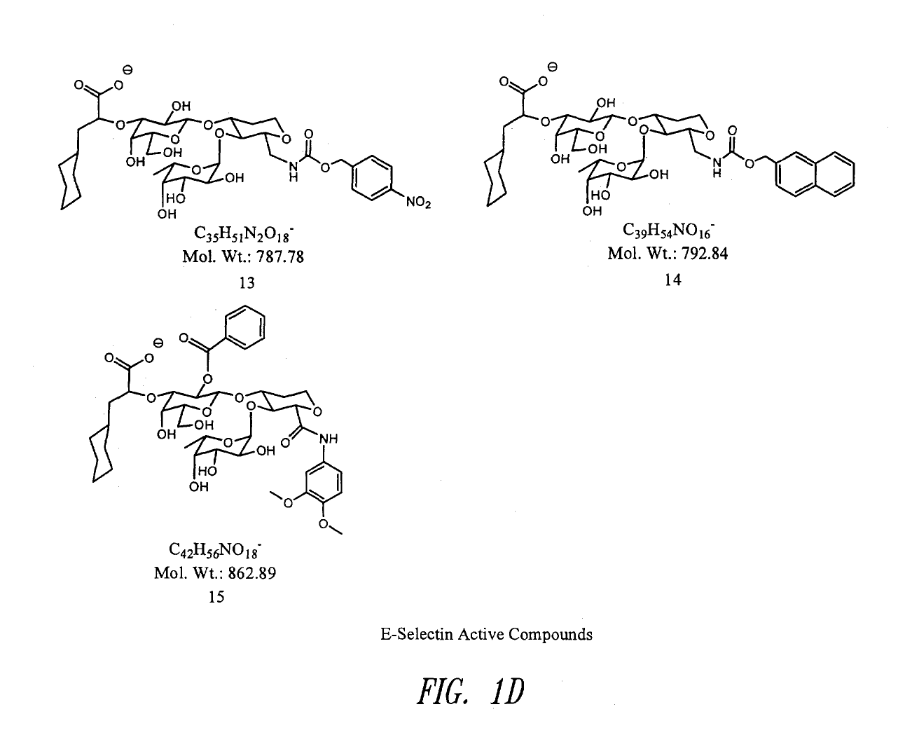

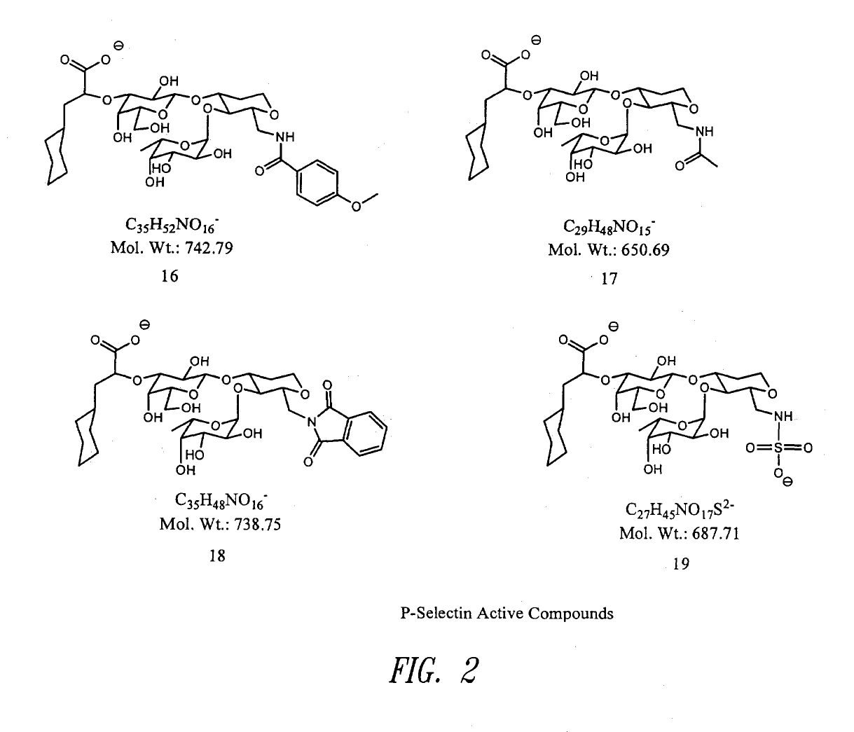

The term “E-selectin compound,” as used herein, refers to a molecule that binds specifically to E-selectin without binding to P-selectin at levels below 10 mM as measured by the ELISA assays described in Example 6. The structures of E-selectin compounds for use in the present invention are shown in FIG. 1. All compounds useful in the present invention, such as E-selectin compounds or conjugates thereof, include physiologically acceptable salts thereof.

For certain embodiments, it may be beneficial to also, or alternatively, have an E-selectin compound possess an additional component or agent, e.g., a detectable component or a therapeutic agent. As used herein, the term “possess” means that a detectable component or therapeutic agent may be covalently or noncovalently bonded to an E-selectin compound either directly or indirectly via one or more other molecules. As used herein, the term “therapeutic agent” refers to any bioactive agent intended for administration to a warm-blooded animal to treat (including prevent) a disease or other undesirable condition or to enhance the success of tissue engineering therapies. Therapeutic agents include drugs, hormones, growth factors, proteins, peptides, genes, viral and non-viral vectors and other compounds. In an embodiment for promoting angiogenesis (e.g., in tissue engineering or medical conditions where angiogenesis is reduced or absent), an E-selectin compound will typically possess an angiogenesis promoting agent. Angiogenesis promoting agents include vascular endothelial growth factor (VEGF), basic and acidic fibroblast growth factor (bFGF and aFGF), transforming growth factor (TGF-β), tumor necrosis factor (TNF-α), hepatocyte growth factor (HGF), angiogenin, interleukin-8 (IL-8) and platelet-derived growth factor (PDGF). In an embodiment for inhibiting angiogenesis (e.g., in tumor growth or medical applications where angiogenesis is occurring), an E-selectin compound will typically possess an angiogenesis inhibiting agent. Angiogenesis inhibiting agents include heparin, suramin, angiostatin, endostatin, alpha and beta interferon, interleukin 12, soluble Flt-1, platelet factor-4, and thrombospondin -1 and -2.

Where an additional component or agent is covalently attached to an E-selectin compound, a conjugate is formed. The phrase “covalently attached” as used herein, refers to both direct attachment and indirect attachment wherein one or more atoms are interposed between an E-selectin compound and an additional component or agent. The one or more interposed atoms may serve to improve the biological properties of the conjugate (e.g., by changing the spatial relationships of the constituents of the conjugate) or to facilitate attachment of the constituents to form the conjugate.

The attachment of a component or agent to an E-selectin compound can be accomplished in a variety of ways to form a conjugate of the present invention. The simplest attachment method is reductive amination of a component or agent to the E-selectin compound's carbohydrate reducing end. This is accomplished by simple reaction of the component or agent to the reducing carbohydrate moiety and subsequent reduction of the imine formed. The loss of the cyclic nature of the sugar reacted with, limits the usefulness of this method. The most general approach entails the simple attachment of an activated linker to the carbohydrate moiety via an O, S or N heteroatom (or C atom for C-linked glycosides) at the anomeric position of the glycan. The methodology of such attachments has been extensively researched and anomeric selectivity is easily accomplished by proper selection of methodology and/or protecting groups. Examples of potential glycosidic synthetic methods include Lewis acid catalyzed bond formation with halogen or peracetylated sugars (Koenigs Knorr), trichloroacetamidate bond formation, thioglycoside activation and coupling, glucal activation and coupling, n-pentenyl coupling, phosphonate ester homologation (Horner-Wadsworth-Emmons reaction), and many others. Alternatively, linkers could be attached to positions on the carbohydrate moieties other than the anomeric. The most accessible site for attachment is at the six hydroxyl (6-OH) position of the sugar (a primary alcohol). The attachment of a linker at the 6-OH can be easily achieved by a variety of means. Examples include reaction of the oxy-anion (alcohol anion formed by deprotonation with base) with an appropriate electrophile such as an alkyl/acyl bromide, chloride or sulfonate ester, activation of the alcohol via reaction with a sulfonate ester chloride or POCl₃ and displacement with a subsequent nucleophile, oxidation of the alcohol to the aldehyde or carboxylic acid for coupling, or even use of the Mitsunobu reaction to introduce differing functionalities. Once attached the carbohydrate linker is then functionalized for reaction with a suitable nucleophile on the E-selectin compound (or vice versa). This is often accomplished by use of thiophosgene and amines to make thiourea-linked heterobifunctional ligands, diethyl squarate attachment and/or simple alkyl/acylation reactions. Additional methods that could be utilized include FMOC solid or solution phase synthetic techniques amenable for carbohydrate and peptide coupling and chemo-enzymatic synthesis techniques possibly utilizing glycosyl/fucosyl transferases and/or oligosaccharyltransferase (OST).

The discussion herein regarding E-selectin compounds (including uses, etc.) applies also to conjugates thereof.

### E-Selectin Compound Formulations

E-selectin compounds or conjugates as described herein may be present within a pharmaceutical composition. A pharmaceutical composition comprises one or more E-selectin compounds or conjugates in combination with one or more pharmaceutically or physiologically acceptable carriers, diluents or excipients. Such compositions may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) or preservatives. Within yet other embodiments, compositions of the present invention may be formulated as a lyophilizate. Compositions of the present invention may be formulated for any appropriate manner of administration, including for example, topical, oral, nasal, intravenous, intracranial, intraperitoneal, subcutaneous, or intramuscular administration.

A pharmaceutical composition may also, or alternatively, contain one or more active agents, such as drugs, which may be linked to an E-selectin compound or may be free within the composition.

The compositions described herein may be administered as part of a sustained release formulation (i.e., a formulation such as a capsule or sponge that effects a slow release of modulating agent following administration). Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of modulating agent release. The amount of E-selectin compound or conjugate contained within a sustained release formulation depends upon the site of implantation, the rate and expected duration of release and the nature of the condition to be treated or prevented.

E-selectin compounds or conjugates are generally present within a pharmaceutical composition in a therapeutically effective amount. A therapeutically effective amount is an amount that results in a discernible patient benefit, such as a measured or observed response of a condition associated with angiogenesis, as described below.

### E-Selectin Compounds Methods of Use

In general, E-selectin compounds or conjugates described herein may be used for achieving diagnostic or therapeutic results in conditions including human disease, or in tissue engineering applications, involving angiogenesis. Such diagnostic or therapeutic results may be achieved in vitro or in vivo, preferably in a warm-blooded animal such as a human, provided that E-selectin is ultimately contacted with an E-selectin compound or conjugate, in an amount and for a time sufficient to achieve a discernible diagnostic or therapeutic result. In the context of this invention, a therapeutic result would relate to the control of the angiogenic process. In some conditions, therapeutic results would be associated with inhibiting angiogenesis. In other conditions, a therapeutic result would require the promotion of angiogenesis, and further may be coupled with the ability to limit neovascularization when the desired therapeutic benefit is achieved.

As used herein, a condition “requires or is associated with angiogenesis” if the condition is characterized by the expression of E-selectin. Such conditions include, for example, cancer, rheumatoid arthritis, wound healing, psoriasis, macular degeneration and diabetic retinopathy, or applications of tissue engineering.

E-selectin compounds or conjugates of the present invention may be used to screen for a condition requiring or associated with angiogenesis. A diagnostically effective amount of an E-selectin compound or conjugate is administered to the patient. The E-selectin compound or conjugate is then detected after a time sufficient to achieve a discernible diagnostic result. In one embodiment, the compound or conjugate possesses a detectable component (i.e., detectable to a person or a machine). As described above, the detectable component may be directly or indirectly (i.e., via one or more other molecules) bound to an E-selectin compound. Alternatively, an E-selectin compound may include a component that is capable of binding a compound which includes a detectable component. This permits the E-selectin compound to be administered independent of a detectable component. For example, numerous pretargeting methodologies are well known in the art (e.g., U.S. Pat. No. 4,863,713 to Goodwin et al. and subsequent improvements by others) wherein the binding partner for a molecule on a cell (in the present invention this is an E-selectin compound for E-selectin on a cell) is delivered to the cell prior to the introduction of a detectable component or therapeutic agent. Generally, a ligand/anti-ligand pair is utilized wherein the ligand is attached to the binding partner, and the anti-ligand is attached to the detectable component or therapeutic agent. A typical ligand/anti-ligand pair is avidin (or streptavidin) and biotin. Pretargeting methodologies are generally used in a two or three step process, and may additionally include a clearing agent other than anti-ligand to remove binding partner-ligand (e.g., E-selectin compound-ligand) not bound to target (e.g., E-selectin). In a preferred embodiment, the detectable component is a radioisotope. The radioisotope may be attached, for example, directly to an E-selectin compound or indirectly to the E-selectin compound (e.g., metal radionuclide which is bound to a chelating compound that is attached to the E-selectin compound). Alternatively, as in a pretargeting methodology, the radioisotope may be attached directly or indirectly to an anti-ligand (and the ligand attached to an E-selectin compound).

E-selectin compounds or conjugates of the present invention may be administered in a manner appropriate to the disease to be treated or to the tissue engineering therapy. As used herein, the term “treat” may include the arrest of cell growth, the killing of cells, the prevention of cells or cell growth, the delay of the onset of cells or cell growth, or the prolongation of survival of an organism. Appropriate dosages and a suitable duration and frequency of administration may be determined by such factors as the condition of the patient, the type and severity of the patient's disease and the method of administration. In general, an appropriate dosage and treatment regimen provide the compound or conjugate in an amount sufficient to provide therapeutic or prophylactic benefit. Within particularly preferred embodiments of the invention, an E-selectin compound or conjugate may be administered at a dosage ranging from about 0.001 to 1000 mg/kg body weight, on a regimen of single or multiple daily doses. Appropriate dosages may generally be determined using experimental models or clinical trials. In general, the use of the minimum dosage that is sufficient to provide effective therapy is preferred. Patients may generally be monitored for therapeutic effectiveness using assays suitable for the condition being treated (including prevention), which will be familiar to those of ordinary skill in the art.

E-selectin compounds may also be used to target substances to cells that express E-selectin. Such substances include therapeutic agents and diagnostic agents. Therapeutic agents may be a molecule, virus, viral component, gene, cell, cell component or any other substance that can be demonstrated to modify the properties of a target cell so as to provide a benefit for treating or preventing a disorder or regulating the physiology of a patient. A therapeutic agent may also be a prodrug that generates an agent having a biological activity in vivo. Molecules that may be therapeutic agents may be, for example, protein, peptides, amino acids, nucleic acids, polynucleotides, steroids, polysaccharides or inorganic compounds. Such molecules may function in any of a variety of ways, including as enzymes, enzyme inhibitors, hormones, receptors, antisense oligonucleotides, catalytic polynucleotides, anti-viral agents, anti-tumor agents, anti-bacterial agents, immunomodulating agents and cytotoxic agents (e.g., radionuclides such as iodine, bromine, lead, palladium or copper). Examples of potential therapeutic agents include antineoplastic agents (such as 5-fluorouracil and distamycin), integrin agonist/antagonists (such as cyclic-RGD peptide), cytokine agonist/antagonists, histamine agonist/antagonists (such as diphenhydramine and chlorpheniramine), antibiotics (such as aminoglycosides and cephalosporins) and redox active biological agents (such as glutathione and thioredoxin). The therapeutic agent may inhibit angiogenesis or promote angiogenesis. Diagnostic agents include imaging agents such as metals and radioactive agents (e.g., gallium, technetium, indium, strontium, iodine, barium, bromine and phosphorus-containing compounds), contrast agents, dyes (e.g., fluorescent dyes and chromophores) and enzymes that catalyze a colorimetric or fluorometric reaction. In general, the possession by an E-selectin compound of a diagnostic or therapeutic agent may be accomplished using a variety of techniques such as those described above. Alternatively, a pretargeting approach (discussed above) may be used (e.g., an E-selectin compound is attached to one member of a ligand/antiligand pair, and a diagnostic or therapeutic agent is attached to the other member). For targeting purposes, an E-selectin compound or conjugate may be administered to a patient as described herein.

E-selectin compounds or conjugates may also be used in vitro, within a diagnostic method, or a variety of well known cell culture and cell separation methods. For example, an E-selectin compound or conjugate may be attached to the interior surface of a tissue culture plate or other cell culture support, for use in immobilizing E-selectin-expressing cells for screens, assays and growth in culture. Such attachment may be performed by any suitable technique, such as the methods described above, as well as other standard techniques. E-selectin compounds or conjugates may also be used to facilitate cell identification and sorting in vitro, permitting the selection of cells expressing E-selectin (or different E-selectin levels). Preferably, the E-selectin compound(s) for use in such methods possess a detectable marker. Suitable markers are well known in the art and include radionuclides, luminescent groups, fluorescent groups, enzymes, dyes, constant immunoglobulin domains and biotin. Within one preferred embodiment, an E-selectin compound possessing a fluorescent marker, such as fluorescein, is contacted with the cells, which are then analyzed by fluorescence activated cell sorting (FACS).

Such in vitro methods generally comprise contacting a biological preparation with any one of compounds 1-15 of FIG. 1, and detecting the compound in the preparation. If desired, one or more wash steps may be added to a method. For example, subsequent to contacting a biological preparation with an E-selectin compound but prior to detection of the compound, the preparation may be washed (i.e., contacted with a fluid and then removal of the fluid in order to remove unbound E-selectin compound). Alternatively, or in addition, a wash step may be added during the detection process. For example, if an E-selectin compound possesses a marker that can bind to a substance that is detectable, it may be desirable to wash the preparation subsequent to contacting the biological preparation with a detectable substance, but prior to the detection. As used herein, the phrase “detecting the compound in the preparation” includes detecting the compound while it is bound to the preparation or detecting the compound which was bound to the preparation but after it has been separated from the preparation.

The following Examples are offered by way of illustration and not by way of limitation.

### EXAMPLES

The syntheses of the E-selectin active compounds within the present invention and the P-selectin active compounds are illustrated in the following references: Helvetica Chemica Acta Vol. 83, pp. 2893-2907 (2000) and Angew. Chem. Int. Ed. Vol. 40, No. 19, pp. 3644-3647 (2001).

### Example 1

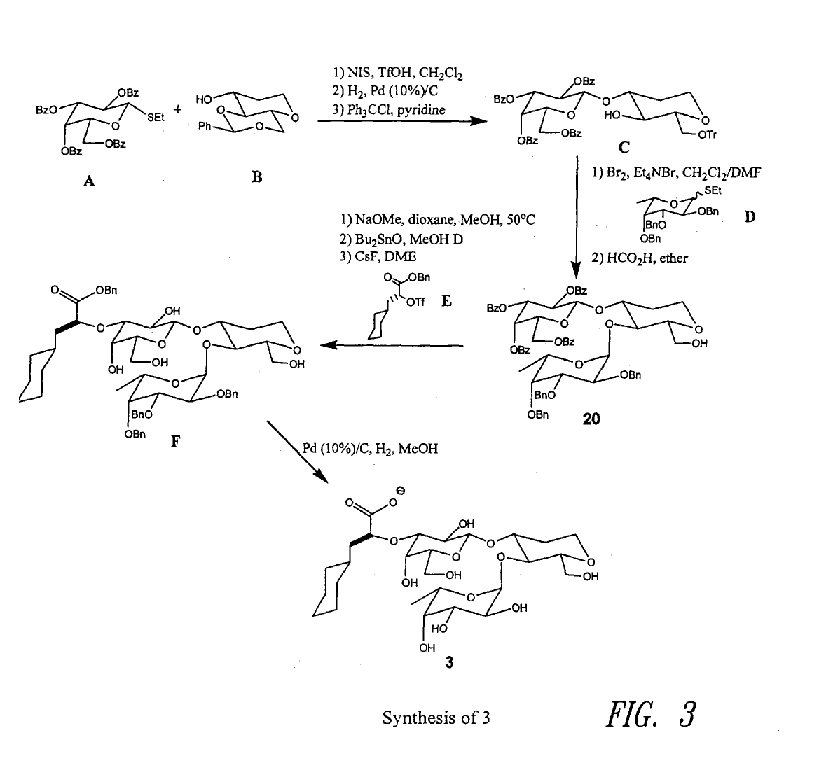

### Synthesis of Compound 3 (FIG. 3)

Formation of Intermediate C:

Compound A (5.00 g, 12.74 mmol) and compound B (4.50 g, 19.11 mmol) and NIS (3.58 g, 15.93 mmol) are dissolved in CH₂Cl₂ (50 ml) and cooled to 0° C. A solution of trifluoromethanesulfonic acid (0.15 M in CH₂Cl₂) is added dropwise with stirring. After the solution changes color from orange to dark brown addition of TMS-OH ceases. The solution is then washed with saturated NaHCO₃ (30 ml) and the organic layer is dried with Na₂SO₄ and evaporated to dryness. The syrup obtained is purified by silica gel chromatography (hexane/ether, 1:1) and used in the next step.

The compound obtained previously is dissolved in THF (40 ml) and Pd (10%)/C (1/10 by mass) is added. The solution is degassed and an atmosphere of H₂ is generated. The reaction is allowed to proceed at RT until disappearance of starting material is confirmed by TLC. The solution is filtered thru a bed of celite and the filtrate is concentrated in vacuo giving the 4 and 6 OH compound. The compound is then dissolved in pyridine (25 ml) and cooled to 0° C. Ph₃CCl (1.2 eq) is added dropwise and the reaction is allowed to proceed at RT for 6 hrs. Ethyl acetate (50 ml) is then added and the solution is washed with 0.1N HCl (2×50 ml), saturated NaHCO₃ (1×50 ml) and saturated NaCl (1×50 ml). The organic layer is dried with Na₂SO₄ and evaporated to dryness. Intermediate C is obtained by silica gel chromatography.

Formation of Compound 20:

Compound C (800 mg, 1.41 mmol) and Et₄NBr (353 mg, 1.69 mmol) are dissolved in DMF/CH₂Cl₂ (10 ml, 1:1, containing molecular sieves) and cooled to 0° C. Br₂ (298 mg, 1.86 mmol, in CH₂Cl₂) is added dropwise to a separate solution of compound D (808 mg, 1.69 mmol) in CH₂Cl₂ at 0° C. After 30 min the Br₂/D solution is quenched with cyclohexene (0.2 ml) and added to the C solution immediately (within 10 min). This mixture is allowed to react for 65 hrs at RT. Ethyl acetate (100 ml) is added, the solution filtered, and the filtrate is washed with saturated NaS₂O₃ (2×50 ml) and saturated NaCl (2×50 ml). The organic layer is dried with Na₂SO₄ and evaporated to dryness. The resultant syrup is then dissolved in ether (50 ml) and formic acid (10 ml), is added with stirring. Upon completion of the reaction (as verified by TLC), the solution is washed with saturated NaHCO₃ (2×50 ml) and saturated NaCl (1×50 ml). The organic layer is dried with Na₂SO₄ then evaporated to dryness. Compound 20 is then purified by silica gel chromatography.

Formation of Intermediate F:

Compound 20 (1 g, 1.02 mmol) is dissolved in MeOH/dioxane (10 ml, 20:1) and NaOMe (0.10 mmol) is added with stirring. The reaction is allowed to proceed at 50° C. for 20 hrs and then 2 drops of acetic acid are added. The solution is evaporated to dryness, dissolved in ethyl ether (25 ml) and washed with saturated NaCl (1×50 ml). The organic layer is dried with Na₂SO₄ and evaporated to dryness. The final product is purified by silica gel chromatography. The product (0.980 mmol) and Bu₂Sn (1.08 mmol) are suspended in MeOH (15 ml) and heated to reflux for 2 hrs. The resultant clear solution is then evaporated to dryness, taken up in pentane (10 ml) and evaporated giving a colorless foam. The foam is dissolved in 1,2-dimethoxyethane (DME, 15 ml), compound E (1.96 mmol) and CsF (1.18 mmol) are added and the reaction stirred for 2 hrs at room temperature. After 2 hrs 1M KH₂PO₄ (50 ml) and KF (1 g) are added with stirring followed by extraction with ethyl acetate (2×25 ml). The organic layer is washed with 10% KF (2×50 ml) and saturated NaCl (2×50 ml), dried with Na₂SO₄ and evaporated to dryness under reduced pressure. Compound F is obtained via silica gel chromatography.

Formation of Compound 3:

Compound F is dissolved in CH₃OH (50 ml) and Pd (10%)/C (1/10 by mass) is added. The solution is degassed and an atmosphere of H₂ is generated. The reaction is allowed to proceed at RT until disappearance of starting material is confirmed by TLC. The solution is filtered thru a bed of celite and the filtrate is concentrated in vacuo giving compound 3.

### Example 2

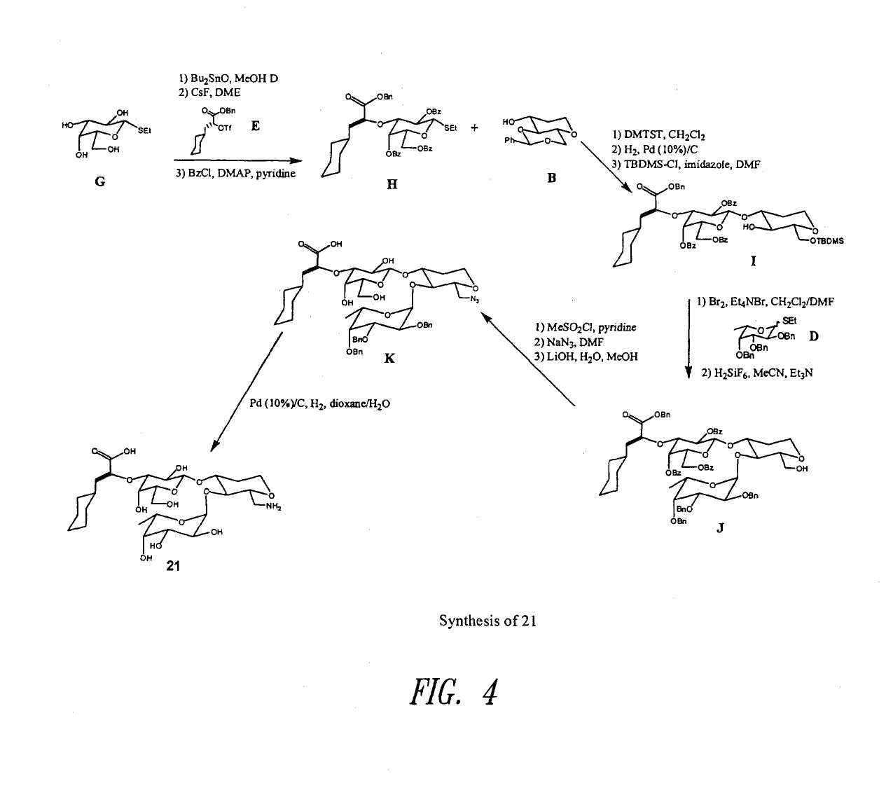

### Synthesis of Compound 21 (FIG. 4)

Formation of Intermediate H:

Compound G (15.0 g, 66.9 mmol) and Bu₂SnO (20.0 g, 80.3 mmol) are suspended in MeOH (450 ml) and heated to reflux for 2 hrs. The resultant clear solution is then evaporated to dryness, taken up in pentane and evaporated again giving a colorless foam. The foam is dissolved in 1,2-dimethoxyethane (DME, 120 ml), E (39.6 g, 100.3 mmol) and CsF (12.2 g, 80.3 mmol) are added and the reaction stirred for 2 hrs at room temperature. After 2 hrs 1M KH₂PO₄ (700 ml) and KF (25 g) are added with stirring followed by extraction with ethyl acetate (3×250 ml). The organic layer is washed with 10% KF (2×250 ml) and sat. NaCl (1×250 ml), dried with Na₂SO₄ and evaporated to dryness under reduced pressure. The compound (19.3 g, 41.2 mmol) is purified by silica gel chromatography and immediately dissolved in pyridine (210 ml) with a crystal DMAP. The solution is cooled to 0° C. and benzoyl chloride (52.1 g, 370.7 mmol) is added dropwise with stirring. The solution is allowed to warm to room temperature slowly and the reaction proceeds at RT for 20 min. The solution is evaporated to dryness, dissolved in ethyl acetate (500 ml), and washed with 0.1M HCl (2×250 ml), saturated NaHCO₃ (2×250 ml) and saturated NaCl (1×250 ml) solutions. The organic layer is dried with Na₂SO₄ and evaporated to dryness. H is obtained via silica gel chromatography.

Formation of Intermediate I:

Intermediate H (10.0 g, 12.82 mmol) and intermediate B (6.05 g, 25.64 mmol) are dissolved in CH₂Cl₂ (75 ml) and 0.15M CF₃SO₃H (in CH₂Cl₂) is added dropwise at −10° C. with stirring. Addition is stopped when the orange solution changes to brown. Ethyl acetate (500 ml) is added and the solution is washed with saturated NaHCO₃ (4×250 ml) and saturated NaCl (250 ml). The organic layer is then dried with Na₂SO₄ and evaporated under reduced pressure. The compound (7.96 g, 9.19 mmol) is then purified by silica gel chromatography and then dissolved in DMF (55 ml). TBDMS-Cl (1.52 g, 10.1 mmol) and imidazole (0.94 g, 13.8 mmol) are then added and the reaction allowed to proceed at RT for 1 hr. Ethyl acetate (250 ml) is added and the solution washed with saturated NaHCO₃ (5×250 ml) and saturated NaCl (1×250 ml). The organic layer is then dried with Na₂SO₄ and purified by silica gel chromatography giving intermediate I.

Formation of Intermediate J:

Compound I (7.71 g, 7.87 mmol) and Et₄NBr (2.00 g, 9.45 mmol) are dissolved in DMF/CH₂Cl₂ (60 ml, 1:1, containing molecular sieves—12 g) and cooled to 0° C. Br₂ (1.90 g, 11.8 mmol) in CH₂Cl₂ (11 ml) is added dropwise to a separate solution of compound D (4.5 g, 9.45 mmol) in CH₂Cl₂ at 0° C. After 30 min the Br₂/D solution is quenched with cyclohexene (2.5 ml) and added to the I solution immediately (within 10 min). This mixture is allowed to react for 65 hrs at RT. CH₂Cl₂ (250 ml) is added, the solution filtered, and the filtrate is washed with saturated NaHCO₃ (2×50 ml), 0.5M HCl (2×250 ml) and saturated NaCl (2×250 ml). The organic layer is dried with Na₂SO₄ and evaporated to dryness. The mixture is dissolved in MeCN (85 ml) at RT and a solution of Et₃N (0.21 ml) and H₂SiF₆ (1.3 ml, 35%) in MeCN (17 ml) is added and stirred for 2 hrs. CH₂Cl₂ (250 ml) is added and the solution washed with saturated NaHCO₃ (3×250 ml) and saturated NaCl (1×250 ml). The organic layer is dried with Na₂SO₄, evaporated to dryness and J is purified by silica gel chromatography.

Formation of Intermediate K:

Intermediate J (12.5 g, 9.75 mmol) is dissolved in pyridine (80 ml) and methanesulfonylchloride (3.35 g, 29.2 mmol) is added dropwise with stirring over 5 min. The reaction is allowed to proceed for 30 min and then ethyl acetate (500 ml) is added. The solution is washed with 1N HCl (250 ml). The organic layer is dried with Na₂SO₄ and evaporated. The resultant syrup (12.95 g, 9.52 mmol) is dissolved in DMF (40 ml) and NaN₃ (4.64 g, 74.4 mmol) is added. The reaction is allowed to proceed for 35 hrs under argon atmosphere at 65° C. The solution is diluted with ethyl acetate (500 ml) and washed with H₂O (300 ml) and saturated NaCl (150 ml). The organic layer is dried with Na₂SO₃ and evaporated to dryness. The compound is purified by silica gel chromatography. The purified product (12.2 g, 9.33 mmol) is then suspended in MeOH/H₂O (200 ml/20 ml) solution and LiOH—H2O (5.1 g, 121.3 mmol) is added. The reaction is allowed to proceed at 65° C. for 20 hrs. Ethyl ether (500 ml) is added and the solution is washed with saturated NaCl (200 ml). The organic layer is dried with Na₂SO₄ and evaporated to dryness. Compound K is purified via silica gel chromatography.

Formation of Compound 21:

Compound K (8.45 g, 9.33 mmol) is dissolved in dioxane/H₂O (250 ml/50 ml) and Pd (10%)/C (3.4 g) is added. The solution is degassed and an atmosphere of H₂ is generated. The reaction is allowed to proceed at RT until disappearance of starting material is confirmed by TLC. The solution is filtered thru a bed of celite and the filtrate is concentrated in vacuo giving compound 21.

### Example 3

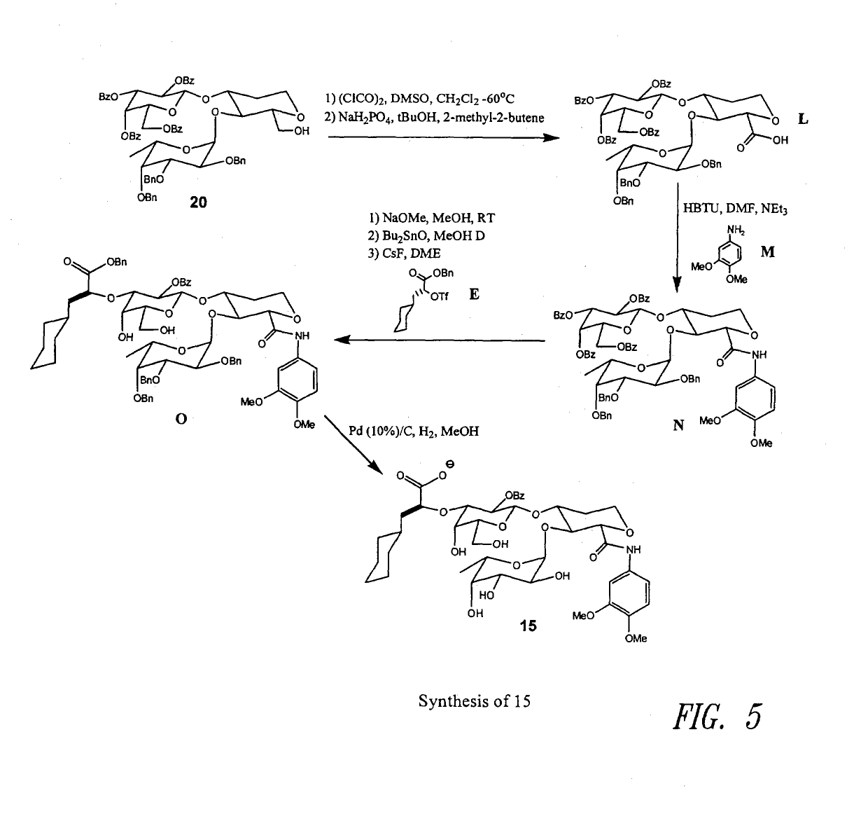

### Synthesis of Compound 15 (FIG. 5)

Formation of Intermediate L:

Compound 20 (10 mmol) is dissolved in CH₂Cl₂ (30 ml) and DMSO (20 mmol) is added and the solution is cooled to −60° C. Oxalyl chloride (11 mmol) is added slowly to the stirred solution of 20. The reaction is allowed to proceed for 30 min under N₂ atmosphere. The reaction is washed with 0.1M HCl, saturated NaHCO₃, and saturated NaCl. The organic layer is dried with Na₂SO₄ and evaporated to dryness. The resultant syrup is placed in tBuOH (20 ml) and 2-methyl-2-butene (10 ml) and NaH₂PO₄ (20 mmol) is added with stirring. The reaction is allowed to proceed for 3 hrs and is then evaporated taken up in CH₂Cl₂ and washed with 0.1M HCl, saturated NaHCO₃, and saturated NaCl. The resultant compound is purified by silica gel chromatography giving compound L.

Formation of Intermediate N:

Compound L (10 mmol) is dissolved in DMF (15 ml) and compound M (10 mmol), HBTU (12 mmol) and Et₃N (20 mmol) are added with stirring. The reaction is allowed to proceed at RT for 24 hrs. Ethyl acetate (100 ml) is added and the solution is washed with 0.1M HCl (1×100 ml), saturated NaHCO₃ (1×100 ml), and saturated NaCl (1×100 ml). The organic layer is dried with Na₂SO₄ and evaporated to dryness. Compound N is isolated via silica gel chromatography.

Formation of Intermediate O:

Compound N (10 mmol) is dissolved in MeOH (35 ml) and NaOMe (1 mmol) is added with stirring. The reaction is allowed to proceed at RT for 20 hrs. The solution is evaporated to dryness, dissolved in ethyl ether (50 ml) and washed with saturated NaCl (1×50 ml). The organic layer is dried with Na₂SO₄ and evaporated to dryness. The final product is purified by silica gel chromatography. The product (0.980 mmol) and Bu₂Sn (1.08 mmol) are suspended in MeOH (15 ml) and heated to reflux for 2 hrs. The resultant clear solution is then evaporated to dryness, taken up in pentane (10 ml) and evaporated giving a colorless foam. The foam is dissolved in 1,2-dimethoxyethane (DME, 15 ml), compound E (1.96 mmol) and CsF (1.18 mmol) are added and the reaction stirred for 2 hrs at room temperature. After 2 hrs 1M KH₂PO₄ (50 ml) and KF (1 g) are added with stirring followed by extraction with ethyl acetate (2×25 ml). The organic layer is washed with 10% KF (2×50 ml) and saturated NaCl (2×50 ml), dried with Na₂SO₄ and evaporated to dryness under reduced pressure. Compound O is obtained via silica gel chromatography.

Formation of Compound 15:

Compound O (9 mmol) is dissolved in MeOH (200 ml) and Pd (10%)/C (3 g) is added. The solution is degassed and an atmosphere of H₂ is generated. The reaction is allowed to proceed at RT until disappearance of starting material is confirmed by TLC. The solution is filtered thru a bed of celite and the filtrate is concentrated in vacuo giving compound 15.

### Example 4

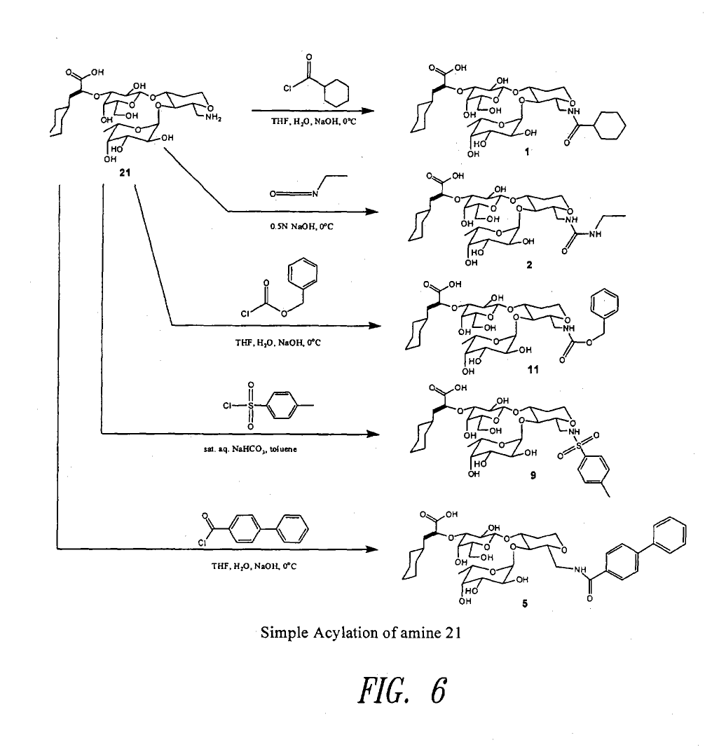

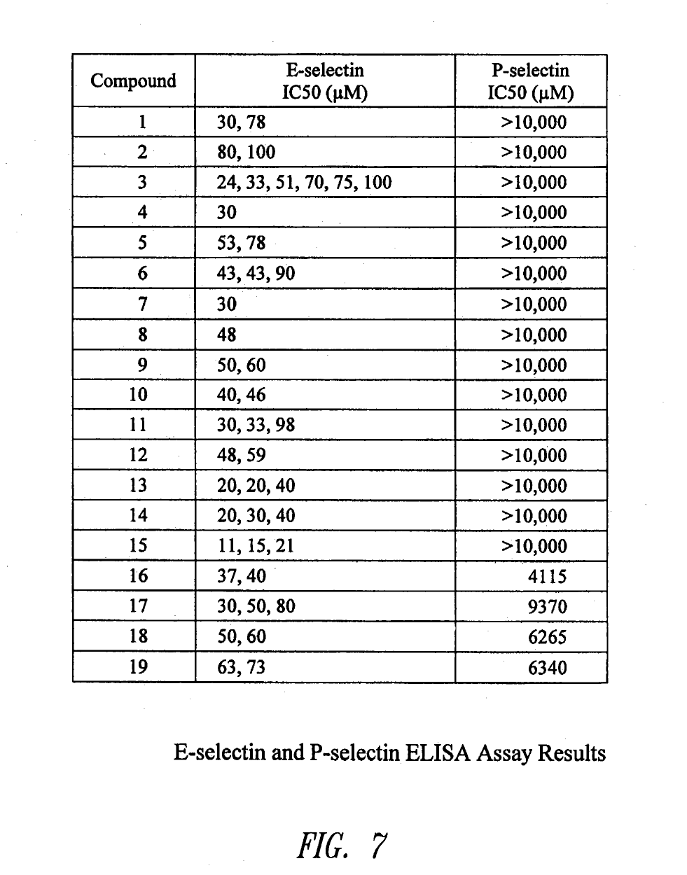

### Acylation of Compound 21 (FIG. 6)

Reaction of Compound 21 with Acid Chlorides:

Compound 21 (20 mg, 0.033 mmol) is dissolved in a THF/H₂O (2 ml, 1:1) solution containing 1N NaOH (pH adjusted between 8-10) and is cooled to 0° C. Cyclohexyl-carbonylchloride (0.049 mmol) is then added dropwise with stirring. The reaction is allowed to continue at 0° C. for 3 hrs. The solution is quenched with ice and the solution is evaporated to dryness. Compound 1 is purified by reverse phase chromatography.

Reaction of Compound 21 with Isocyanates:

Compound 21 (30 mg, 0.049 mmol) is dissolved in a 0.5N aqueous NaOH solution (1 ml) and cooled to 0° C. Ethyl isocyanate (1.2 eq) is then added dropwise with stirring. The reaction is allowed to continue at RT for 3 hrs. The solution is quenched with ice and the solution is evaporated to dryness. Compound 2 is purified by reverse phase chromatography.

Reaction of Compound 21 with Chloro-Orthoformates:

Compound 21 (20 mg, 0.033 mmol) is dissolved in a THF/H₂O (2 ml, 1:1) solution containing NaOH (pH adjusted between 8-10) and is cooled to 0° C. Benzyl-chloro-orthoformate (0.049 mmol) is then added dropwise with stirring. The reaction is allowed to continue at 0° C. for 3 hrs. The solution is quenched with ice and the solution is evaporated to dryness. Compound 11 is purified by reverse phase chromatography.

Reaction of Compound 21 with Sulfonyl Chlorides:

Compound 21 (20 mg, 0.033 mmol) is dissolved in a saturated aqueous NaHCO₃/toluene (2 ml, 1:1) solution and is cooled to 0° C. p-Toluenesulfonyl chloride (0.049 mmol) is then added dropwise with stirring. The reaction is allowed to continue at 0° C. for 3 hrs. The solution is quenched with ice and the solution is evaporated to dryness. Compound 9 is purified by reverse phase chromatography.

### Example 5

### Assay for E-Selectin Antagonist Activity

Wells of a microtiter plate (plate 1) are coated with E-selectin/hlg chimera (GlycoTech Corp., Rockville, Md.) by incubation for 2 hr at 37° C. After washing the plate 5 times with 50 mM TrisHCl, 150 mM NaCl, 2 mM CaCl₂, pH 7.4 (Tris-Ca), 100 μl of 1% BSA in Tris-Ca/Stabilcoat (SurModics, Eden Prarie, Minn.) (1:1, v/v) are added to each well to block non-specific binding. Test compounds are serially diluted in a second low-binding, round bottomed plate (plate 2) in Tris-Ca (60 μl/well). Preformed conjugates of SLea-PAA-biotin (GlycoTech Corp., Rockville, Md.) mixed with Streptavidin-HRP (Sigma, St. Louis, Mo.) are added to each well of plate 2 (60 μl/well of 1 μg/ml). Plate 1 is washed several times with Tris-Ca and 100 μl/well are transferred from plate 2 to plate 1. After incubation at room temperature for exactly 2 hours the plate is washed and 100 μl/well of TMB reagent (KPL labs, Gaithersburg, Md.) is added to each well. After incubation for 3 minutes at room temperature, the reaction is stopped by adding 100 μl/well of 1M H₃PO₄ and the absorbance of light at 450 nm is determined by a microtiter plate reader.

### Example 6

### Assay for P-Selectin Antagonist Activity

The neoglycoprotein, sialylLe^{a}-HSA (Isosep AB, Sweden) is coated onto wells of a microtiter plate (plate 1) and the wells are then blocked by the addition of 2% bovine serum albumin (BSA) diluted in Dulbecco's phosphate-buffered saline (DPBS). In a second microtiter plate (plate 2), test antagonists are serially diluted in 1% BSA in DPBS. After blocking, plate 1 is washed and the contents of plate 2 are transferred to plate 1. Pselectin/hlg recombinant chimeric protein (GlycoTech Corp., Rockville, Md.) is further added to each well in plate 1 and the binding process is allowed to incubate for 2 hours at room temperature. Plate 1 is then washed with DPBS and peroxidase-labelled goat anti-human Ig(γ) (KPL Labs, Gaithersburg, Md.) at 1 μg/ml is added to each well. After incubation at room temperature for 1 hour, the plate is washed with DBPS and then TMB substrate (KPL Labs) is added to each well. After 5 minutes, the reaction is stopped by the addition of 1M H₃PO₄. Absorbance of light at 450 nm is then determined using a microtiter plate reader.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method of screening in vivo for a condition requiring or associated with angiogenesis, comprising the steps of:
(a) administering to a warm-blooded animal in a diagnostically effective amount any one of compounds 1-15 of FIG. 1; and
(b) detecting the compound in the animal.

2. The method of claim 1 wherein the compound possesses a detectable component.

3. The method of claim 2 wherein the detectable component is a radioisotope.

4. A method of screening in vitro for a condition requiring or associated with angiogenesis, comprising the steps of:
(a) contacting a biological preparation with a diagnostically effective amount of any one of compounds 1-15 of FIG. 1; and
(b) detecting the compound in the preparation.

5. The method of claim 4 wherein the compound possesses a detectable component.

6. The method of claim 5 wherein the detectable component is a fluorescent group.

7. The method of claim 6 wherein the fluorescent group is detected by fluorescence activated cell sorting.

8. A method for in vitro identification of cells expressing E-selectin, comprising the steps of:
(a) contacting a biological preparation with any one of compounds 1-15 of FIG. 1; and
(b) detecting the compound in the preparation.

9. The method of claim 8 wherein the compound possesses a detectable component.

10. The method of claim 9 wherein the detectable component is a fluorescent group.

11. The method of claim 10 wherein the fluorescent group is detected by fluorescence activated cell sorting.

12. A method of treating a condition requiring or associated with angiogenesis, comprising the step of administering to a warm-blooded animal in a therapeutically effective amount any one of compounds 1-15 of FIG. 1.

13. The method of claim 12 wherein the compound possesses a therapeutic agent.

14. The method of claim 13 wherein the therapeutic agent inhibits angiogenesis.

15. The method of claim 13 wherein the therapeutic agent promotes angiogenesis.

16. A method for promoting angiogenesis in tissue engineering, comprising the step of contacting cells with any one of compounds 1-15 of FIG. 1, wherein the compound possesses an angiogenesis promoting agent.

17. A conjugate comprising any one of compounds 1-15 of FIG. 1 covalently attached to a diagnostic or therapeutic agent.

18. The conjugate of claim 17 wherein the therapeutic agent is an antineoplastic agent.

19. The conjugate of claim 17 wherein the therapeutic agent is an angiogenesis promoting agent.

20. The conjugate of claim 17 wherein the therapeutic agent is an angiogenesis inhibiting agent.
